(19) United States
(12) Patent Application Publication
(10) Pub. No.: US 2025/0171863 A1
(43) Pub. Date: May 29, 2025

(54) CROP PATHOGEN MONITORING AND POPULATION PREDICTION

(71) Applicant: SYNGENTA CROP PROTECTION AG, Basel (CH)

(72) Inventors: Stefano TORRIANI, Stein (CH); Sarah Margaret TARGETT, Bracknell, Berkshire (GB); Paolo GALLI, Basel (CH); Luca CORNETTI, Stein (CH); Lorenzo BORGHI, Stein (CH)

(73) Assignee: SYNGENTA CROP PROTECTION AG, Basel (CH)

(21) Appl. No.: 18/730,576

(22) PCT Filed: Jan. 18, 2023
(86) PCT No.: PCT/EP2023/051106

§ 371 Date: Jul. 19, 2024

(30) Foreign Application Priority Data
Jan. 19, 2022 (EP) 22152293.1

(51) Int. Cl. C12Q 1/6895

(52) U.S. Cl. CPC C12Q 1/6895; C12Q 2600/156

(57) ABSTRACT

The present invention relates to a method for identifying a phytopathogen population including variants in a field or greenhouse, the method comprising: a) collecting a set of samples at a location comprising at least part of a harmful phytopathogenic organism; b) processing the sample set; and analyzing cach samples by a whole metagenome sequence analysis, thereby producing whole metagenome reads; c) sequencing DNA and/or RNA of the harmful organism and ascertaining one or more DNA and/or RNA sequences; d) optionally, subjecting the whole metagenome reads to quality control procedures comprising removal of non-phytopathogen reads; e) comparing the remaining reads to one or more k-mer arrays from known fungi by decomposing the reads into a set of sample derived k-mers of about 300 base pairs; and f) analyzing the k-mer mode read tables to determine the biological sample based on comparative k-mer analysis with known fungi subtypes, or other biological samples with known outcome or taxonomic composition; and g) entering information about resistance of the determined phytopathogen population, comprising each individual variety determined in the sample as well as the occurrence of each individual variety into a resistance heatmap, and/or a resistance prediction module.

### TECHNICAL FIELD

The present invention is concerned with the recognition of resistances in harmful organisms to control agents and with the detection of virulence of harmful organisms to resistant crop varieties. Specifically, it relates to computer assisted identification of variants in a population of phytopathogenic organisms, and variations thereof influencing their sensitivity to control agents or their ability to grow on tolerant crops. The present teachings also relate to computer assisted monitor one or more of such organisms, and methods and systems for recognizing and logging resistances. The methods and systems relate to creating a resistance map in which information on the resistance of one or more harmful organisms to one or more control agents is listed for a field or a plurality of fields for the growing of crop plants.

### BACKGROUND

With a growing population needing access to food, it has become increasingly necessary for the crop yields to be increased. Crop protection agents such as fungicides have in the past contributed considerably to the raising of yields, by keeping harmful phytopathogenic organisms at bay. Increasingly, however, resistances are being observed in fungi, causing increasing loss of yields, down to complete destruction of harvests. In addition, regulatory restrictions are limiting the number of plant protection products, such as fungicides, available to the farmer.

To control plant pests, fungicidal chemical or biological compounds and formulations are continuously being developed and improved, in an arms' race with nature, as existing fungi respond to pesticidal chemical or biological compounds by evolving and developing resistance against these compounds, usually by mutation, or by acquiring traits. Fungi are usually prone to mutate and produce a variety which has a high degree of tolerance or are even resistance to the applied pesticidal compound, or even against mixtures of different pesticidal chemical compounds.

Once a variety occurs that is tolerant or resistant, it will be positively discriminated vis-à-vis the non-tolerant predecessor varieties, as the latter are repressed by the active ingredients, allowing tolerant or resistant strains to outgrow the non-tolerant strains. Eventually, the pathogen population will primarily be composed of the now resistant individuals.

In fields or greenhouses with an established tolerant population, the previously applied pesticides thus become ineffective against the resistant organism. Accordingly, application of such a compound to control the resistant organism, and/or other compounds to which cross-resistance exists may eventually become useless.

In order to control resistances and to prevent the spread of resistant harmful fungi, however, it is important to be able to recognize the presence of tolerant or resistant varieties in a field, preferably at an early stage, and to monitor their spread. Also, there is a need to predict the development of certain resistance traits in a population and a given area, and/or to prevent the spread and multiplication by positive discrimination due to inadequate suppression of the parental, less tolerant varieties.

### SUMMARY

As discussed above, plant phytopathogens can develop resistance towards a pesticide such that the pesticide becomes less effective, or even ineffective, in managing the infection, reproduction and spread of the pathogen. Also, virulence of a phytopathogen can change as a result of mutation and also differ depending on the plant variety. As such, disease control and resistance management of pesticides need to be carefully implemented to avoid fast selection of resistance leading to ineffectiveness and diminished options available to a farmer, agriculturalist, agronomist or the like for controlling the pathogen. The present disclosure at least partially aims to address this challenge.

Accordingly, in a first aspect, the present disclosure relates to a method for identifying a phytopathogen population including variants in a field or greenhouse, the method comprising: a) collecting a set of samples at a location comprising at least part of a harmful phytopathogenic organism; b) processing the sample set; and analyzing each sample by a whole metagenome sequence analysis, thereby producing whole metagenome reads; c) sequencing DNA and/or RNA of the harmful organism and ascertaining one or more DNA and/or RNA sequences; d) optionally, subjecting the whole metagenome reads to quality control procedures comprising removal of non-phytopathogen reads; e) comparing the reads, or remaining reads to one or more k-mer arrays from known fungi by decomposing the reads into a set of sample-derived k-mers of 300 base pairs or more; and f) analyzing the k-mer mode read tables to determine the biological sample based on comparative k-mer analysis with known fungi subtypes, or other biological samples with known outcome or taxonomic composition; and g) entering information about resistance of the determined phytopathogen population, comprising each individual variety determined in the sample as well as the occurrence of each individual variety into a resistance heatmap, and/or a resistance prediction module.

In heatmaps, values are identified by using continuous or discrete colour levels. In this way, distribution and a change rule of a variable in two-dimensional space may be demonstrated vividly and naturally. Therefore, a heatmap is applied to various scenarios to present density data and reflect a region feature. For example, when density data of different regions is presented by using colour levels in a heatmap in an electronic map, a “hot region” in which data is dense and a “cold region” in which data is sparse can be clearly presented on the map, so as to demonstrate important statistics such as population distribution.

Currently, in an image with a proper proportional scale, a heatmap is usually drawn according to density data by using a point depiction method. A frequently used drawing method is drawing one heat point for one data point or drawing one heat point for a sum of data in one region. On the heatmap based on this, once the proportional scale of the image changes, the heatmap cannot reflect a region feature continuously and accurately.

Using an example in which a heatmap is applied to an electronic map, a characteristic of the electronic map is that geographical location information is completed and continuous. However, for a heatmap generated by using existing heatmap technologies, when a user zooms in the map, the heatmap is presented as a scatter chart, and the entire scatter chart cannot reflect a distribution rule of density data in different regions.

In some embodiments, the method comprises collecting a set of samples comprising at least part of the population of the harmful organism strains present at a field location; processing the sample set, sequencing DNA and/or RNA of the harmful organisms in each sample, ascertaining one or more DNA and/or RNA sequences of sufficient length to link one or more traits to a single variety; and analyzing the one or more DNA and/or RNA sequences for markers linked to resistance development of the harmful organism to a control agent; and collating qualitative and quantitative information about the presence of one or more resistance traits of harmful organism varieties to the control agent in the test location.

Accordingly, the present disclosure provides means allowing a user to recognize whether a population of a harmful organism has appeared in a region and could develop, is in the process of developing, or has already developed resistance to a control agent.

In a further aspect, the present disclosure also relates to a system for identifying infection in a sample, comprising: one or more processors; and one or more non-transitory computer readable storage media storing computer readable instructions that when executed by the one or more processors cause the processors to perform the method of the invention.

In a further aspect, the present disclosure also relates to a non-transitory computer-readable media for identifying presence of a phytopathogen variety in a sample, the non-transitory computer-readable media storing instructions that when executed cause a computer to perform the method of the invention.

The term “region” refers generally to a spatially de-limitable tract of the Earth's surface. A region may comprise one or more agriculturally exploited or exploitable areas, such as fields or greenhouses, or a geographic domain.

A “harmful organism” is understood as meaning a phytopathogenic organism which acts as a causative organism or transmitter of diseases in plants or crops, or which is capable of appearing when crop plants are grown and of damaging the crop plant, adversely affecting the harvesting of the crop plant, or competing with the crop plant for natural resources.

Examples of harmful organisms are Zymoseptoria tritici, Puccinia recondita and Puccinia striiformis in wheat; Pyrenophora teres, Puccinia hordei and Ramularia collo-cygni in barley; Plasmopara viticola and Uncinula necator in grape; Phakopsora pachyrhizi and Corynespora cassiicola in soybean.

The term “control agent” refers to an agent with which harmful organisms can be effectively controlled and/or their spread prevented. Examples of control agents are herbicides, insecticides, nematicides, acaricides, and fungicides. A control agent typically comprises one or more active ingredients. “Active ingredients” are chemical or biological substances which in an organism have a specific effect and/or evoke a specific response.

In a first step, at least one sample is collected, said sample comprising at least part of a harmful organism. The term “collecting” should not be understood in any way as limiting. One example of a synonymous term is the term “sampling”.

Geolocation may take place with the aid of a mobile device or by means of a mobile device which, for example, moves or is moved in a field for crop plants and/or moves or is moved over the field. Conceivable, for example, is the use of a manned or unmanned machines, for instance airborne or landlocked drones and/or robots.

The nature of the sample is dependent on the harmful organism for which the aim is to examine whether individuals are present which are developing or have developed resistance to a control agent. The sample is typically a harmful organism or part of a harmful organism.

A sample is preferably taken from the infested organism, on which the fungus is located. It is also conceivable to take air, water and/or soil samples in which harmful organisms are located. It may be the case that the reason for the sampling is that a harmful organism has been observed by inspection. It may also be the case that the sampling has taken place as a result of the suspected incidence of the harmful organism. It is conceivable, for example, that, using a forecast model, a risk of infestation with the harmful organism has been ascertained, the risk lying above a defined threshold value. It is conceivable that infestation has been observed in the vicinity of the location at which a sample is collected. It may also be the case that the reason for the sampling is a suspicion of existing or oncoming resistance. It is conceivable, for example, for an observation to have been made, when controlling a harmful organism with a control agent, that the control agent is not developing the desired effect. It may alternatively be the case that the prime reason for the sampling is the charting of resistances.

The representative set of samples is ideally taken at different locations within a field or greenhouse, and preferably, geolocation as well as climate data are registered when the sample is taken at each location. For those locations at which each sample is taken, the geocoordinates associated with the location may advantageously be ascertained. This is important to enable information concerning a resistance to be associated with the corresponding location and entered in a resistance development heat map, as well as for predicting the occurrence of certain varieties. Geocoordinates may advantageously be collated using a positional determination system, e.g., a satellite navigation system such as the NAVSTAR GPS, referred to herein as Global Positioning System (GPS). The location and other data may also be registered by mobile communication devices, such as smartphones, e.g., at a location at which a user is taking a sample, the geocoordinates of the sampling location can be logged using a GPS sensor of the device, as well as e.g. temperature or humidity readings. Where a sampling device is stationary, e.g., spore traps, the position may be recorded when placing the device,

Sample Processing: After a sample has been taken, it is processed. The purpose of the processing is to prepare for subsequent sequencing of DNA and/or RNA. By means of the processing, the sample or part of the sample is therefore processed in such a way that it can be subjected to sequencing.

The corresponding processing measures are well known and described in the prior art. Processing may take place at the same location at which the sample was taken. This means that the sample, after having been collected, will be further processed and sequenced locally. Alternatively, samples may be sent to a laboratory for further processing.

The processing is followed by the sequencing of RNA and/or DNA present in a sample. The purpose of the sequencing is to identify resistance markers, and to associate those with one or more particular pathogen varieties. A “resistance marker” is a piece of genetic information which reveals whether a harmful organism might develop, is developing, or has developed resistance to a control agent. The sequencing and analysis of the sequences serves to identify one or more resistance markers in the sample. The term “resistance” refers to a heritable property of individual harmful organisms of one species that is manifested by these individuals withstanding a treatment with a control agent with which the species can normally be controlled and concluding their lifecycle normally.

The analysis looks at whether there are defined DNA and/or RNA sequences present quantitatively and/or qualitatively in the sample which indicate that the harmful organism might develop, is developing, or has developed resistance to a control agent. In the identification of resistance markers, it is possible to look at whether there are DNA sequences and/or RNA sequences in the sample that are known to be responsible for resistance. In the case of metabolic resistances, moreover, it is possible to use the quantity of the corresponding RNA in the organism as a resistance marker. It is also conceivable that a DNA and/or RNA sequence is identified which coincides neither with the sequences of known resistance markers nor with sequences of the non-resistant harmful organism. This new type of sequence might point to a newly forming resistance and/or might indicate a new resistance marker, whereby DNA sequences and/or RNA sequences may indicate that the harmful organisms have or are developing resistance to a control agent are known for a multiplicity of harmful organisms. A DNA sequence and/or RNA sequence of this kind and/or the quantity thereof is hence suitable as a reference marker in the sense of the present invention.

For the identification of resistance markers in the field on the basis of DNA and/or RNA sequencing, methods have been published, see for instance EP 3 464 624 A1 in the name of Oxford Nanopore Technologies Ltd.

Preferably, performing molecular analysis of the biological sample further comprises obtaining the biological sample from a field suspected of having a fungal infection.

Preferably, performing molecular analysis of the biological sample further comprises isolating total DNA from the biological sample obtained from the sample.

Preferably, performing molecular analysis of the biological sample further comprises removing anon-fungal DNA from the isolated total DNA resulting in a fungal DNA sample.

Preferably, performing molecular analysis of the biological sample further comprises ligating sequencing platform-specific adaptors to the fungal DNA sample.

Preferably, performing molecular analysis of the biological sample further comprises indexing the fungal DNA sample.

Preferably, the method is used for diagnosis and analyzing comprises creating a read mode table by comparing the k-mers derived from each sample read to the k-mer array from known fungal reference sequences and summarizing a read mode table into a presence report.

Preferably, the method is used for prognosis and analyzing comprises creating a read mode table by comparing the k-mers derived from each sample read to the k-mer array from to k-mers derived from sequencing samples with known fungal pathogen varieties and summarizing the read mode table into a sample read abundance matrix.

Preferably, the method further comprises the steps of: extracting sequence reads; and translating the extracted sequence reads into putative protein sequences.

Preferably, the method also further comprises the step of analyzing the putative protein sequences against protein motif databases to identify protein functions that correlate significantly with resistance information.

Preferably, the method also further comprises providing a treatment proposal to as field or greenhouse based upon the prognosis of resistance development.

By means of the sequencing and analysis of the sequences it is possible, therefore, to tell whether an individual of a harmful organism has developed resistance to a control agent or whether genetic alterations are present which indicate that the harmful organism is developing or might develop resistance.

Applicants found that while in the state-of-the art analytical methods, the occurrence of one or more particular resistance alleles or sequences could be determined, it was not or almost not possible to attribute such allele to a particular variety. For instance, if a modification A and a modification B in occurred in 30% of the samples, there was no guarantee those modification or mutations had occurred within one or several different trains of varieties.

Applicants found that however, if the length of the analysed DNA sequences was at least 300 base pairs or longer, i.e. the k-mer was at least 300 base pairs, this permitted to associate those with a particular strain, thereby reducing, or eliminating completely the potential overlap between different strains. Accordingly, with the present method, it is feasible to discern between different varieties of closely related fungi, thereby allowing for a much more precise analysis of the tested population, and to improve prediction of resistance development.

The outcome of the present method may be a reference heat map that may advantageously provide a farmer or grower with valuable information as to the presence or genesis of resistances in particular areas of his field or greenhouses. If repeated, insights are gained into the spread of resistances, and the efficacy of measures can be modelled.

Furthermore, the resultant population maps may be combined with additional data, such as location, temperature, air humidity, and wind direction, and hence permit a precise prediction modelling of the spread of resistant harmful organisms. This allows targeted control, and/or prophylactic treatment of bordering territories, and also a more targeted deployment of control agents.

Furthermore, the targeted logging of genetic information on harmful organisms in the environment allows an estimation of an impending development of resistance to a particular control agent. In some embodiments, a measure is carried out for controlling a harmful organism and/or a (developing) resistance on the basis of the reference map. It is conceivable, for example, for those areas affected by a resistant harmful organism (resistant areas), and preferably also areas adjoining the resistant areas, to be treated with a control agent for which no resistance has been identified.

The reference resistance heatmap may allow associating the one or more at least one genetic variance and corresponding quantity with a level of resistance to a pesticide and/or a group of pesticides. Additionally, or alternatively, generating a virulence profile may comprise associating the at least one genetic variance and corresponding quantity with a level of virulence. This provides a much better overarching understanding of the resistance and/or virulence of a pathogen or plant pathogen population present in a plant or a location such as a field of plants.

The method may further advantageously comprise determining at least one disease control measure for controlling the development, reproduction and/or viability of the plant pathogen based on the resistance profile and/or virulence profile. This is particularly useful for a farmer, agriculturalist, agronomist or the like trying to control a disease present in a field as the disease control measure can be tailored to the specific variant(s) of the pathogen present in the field whilst managing resistance. For example, the determined disease control measure may be at least one or more fungicide, or a combination thereof.

Sequencing of fungal DNA has been described for instance in N. Boonham et al., Eur J Plant Pathol, 121; C. Prassan et al., World J Microbiol Biotechno/37; S. R. Bulman et al., Australas. Plant Pathol. 47, 5; YU L ET AL: Plant Soil 357 (2012), p. 395-405; and I. P. Adams et al., Eur J Plant Pathol 152, (2018), 909-919. However, these documents do either not mention resistance development at all, or only in the context of speculating that further developments of the sequencing technologies may be useful for identifying invasive fungal disease outbreaks. A similar document, H. Van der Heyden et al, Agron. Sustain. Dev. 41, (2021), discloses the detection of airborne fungal spores by DNA sequencing. It also mentions DNA sequencing for Zymoseptoria tritici to monitor specific substitutions associated with resistance to DMI and SDHI active agents. However, there is no teaching on how DNA sequencing could be used to provide information on resistance as set out in the present method.

Accordingly, a particular advantage of the subject method is that sufficiently long DNA sequence reads are obtained, which make it feasible to discern between different varieties of closely related fungi, thereby allowing for a more precise analysis of a tested population, and to improve prediction of resistance development. Furthermore, step g) of the method relates to the content of the data obtained through DNA sequencing and enables a user to recognise whether a population of a harmful organism has appeared in a region and could develop, is in the process of developing, or has already developed resistance to a control agent.

The following, non-limiting example illustrates the invention:

### EXAMPLE 1

A farmer reports pathogen infestation despite treatment with corresponding crop protection agents. Samples of the pathogen are taken in situ by the farmer, together with associated plant material. The samples are then processed and the DNA and/or RNA in the sample is sequenced. The sequence information is analysed and examined for reference markers for metabolic and target site resistances, and time, coordinates of wild type and resistant varieties are recorded in a database for studies on resistance spread. Based on the possible resistance mechanism or mechanisms found at the location and on the surroundings, recommendations for a crop protection product are communicated to the farmer.

1. A method for identifying a phytopathogen population including variants in a field or greenhouse, the method comprising:
a) collecting a set of samples at a location comprising at least part of a harmful phytopathogenic organism;
b) processing the sample set; and analyzing each sample by a whole metagenome sequence analysis, thereby producing whole metagenome reads;
c) sequencing DNA and/or RNA of the harmful organism and ascertaining one or more DNA and/or RNA sequences;
d) optionally, subjecting the whole metagenome reads to quality control procedures comprising removal of non-phytopathogen reads;
e) comparing the remaining reads to one or more k-mer arrays from known fungi by decomposing the reads into a set of sample derived k-mers of about 300 base pairs; and
f) analyzing the k-mer mode read tables to determine the biological sample based on comparative k-mer analysis with known fungi subtypes, or other biological samples with known outcome or taxonomic composition; and
g) entering information about resistance of the determined phytopathogen population, comprising each individual variety determined in the sample as well as the occurrence of each individual variety into a resistance heatmap, and/or a resistance prediction module.

2. The method according to claim 1, wherein performing molecular analysis of the biological sample further comprises obtaining the biological sample from a field suspected of having a fungal infection.

3. The method according to claim 2, wherein performing molecular analysis of the biological sample further comprises isolating total DNA from the biological sample obtained from the sample.

4. The method according to claim 3, wherein performing molecular analysis of the biological sample further comprises removing anon-fungal DNA from the isolated total DNA resulting in a fungal DNA sample.

5. The method according to claim 4, wherein performing molecular analysis of the biological sample further comprises ligating sequencing platform-specific adaptors to the fungal DNA sample.

6. The method according to claim 5, wherein performing molecular analysis of the biological sample further comprises indexing the fungal DNA sample.

7. The method according to claim 1, wherein the method is used for diagnosis and analyzing comprises creating a read mode table by comparing the k-mers derived from each sample read to the k-mer array from known fungal reference sequences and summarizing a read mode table into a presence report.

8. The method according to claim 1, wherein the method is used for prognosis and analyzing comprises creating a read mode table by comparing the k-mers derived from each sample read to the k-mer array from to k-mers derived from sequencing samples with known fundal pathogen varieties and summarizing the read mode table into a sample read abundance matrix.

9. The method according to claim 1, further comprising the steps of: extracting sequence reads; and translating the extracted sequence reads into putative protein sequences.

10. The method according to claim 9, further comprising the step of analyzing said putative protein sequences against protein motif databases to identify protein functions that correlate significantly with resistance information.

11. The method according to claim 1, further comprising providing a treatment proposal to a field or greenhouse based upon the prognosis of resistance development.

12. The method according to claim 1, for use in identifying populations of Zymoseptoria tritici, Puccinia recondita, Puccinia striiformis, Pyrenophora teres, Puccinia hordei, Ramularia collo-cygni, Plasmopara viticola, Uncimda necator, Phakopsora pachyrhizi and/or Corynespora cassiicola.

13. A system for identifying infection in a sample, comprising:
one or more processors; and one or more non-transitory computer readable storage media storing computer readable instructions that when executed by the one or more processors cause the processors to perform the method of claim 1.

14. A non-transitory computer-readable media for identifying presence of a phytopathogen variety in a sample, the non-transitory computer-readable media storing instructions that when executed cause a computer to perform the method of claim 1.
